(19) United States
(12) Plant Patent Application Publication
(10) Pub. No.: US 2015/0052645 P1
(43) Pub. Date: Feb. 19, 2015

(54) Sweet Orange Tree Named 'OLL-8'

(50) ;

(71) Applicant: Florida Foundation Seed Producers, Inc., Marianna, FL (US)

(72) Inventor: Jude W. Grosser, Winter Haven, FL (US)

(73) Assignee: Florida Foundation Seed Producers, Inc., Marianna, FL (US)

(21) Appl. No.: 13/987,609

(22) Filed: Aug. 13, 2013

(51) Int. Cl. A01H 5/00

(52) U.S. Cl. PLT/202

(57) ABSTRACT

A new and distinct sweet orange clone ‘OLL-8’ has been developed from an unstable sweet orange selection, designated as ‘OLL’, using tissue culture techniques. Undeveloped ovules from fruit of selection ‘OLL’ were cultured in vitro to generate an embryogenic callus, from which multiple independent clones were regenerated via somatic embryogenesis. Regenerated plantlets (somaclones) were grafted to Swingle citrumelo rootstock and grown in the field for evaluation. Sweet orange clone ‘OLL-8’ was the most precocious bearing clone among the somaclone population, and thus the first to be selected for release, based on its stable propagation and outstanding qualities. ‘OLL-8’ produces high yields of high quality fruit, with exceptional flavor and juice color. It is expected to make a significant contribution to the sweet orange portfolio available to the not-from-concentrate juice producers in Florida and around the world. It also has potential as a fresh fruit cultivar, making it a dual-use fruit.

### LATIN NAME OF THE GENUS AND SPECIES OF THE PLANT CLAIMED

Citrus sinensis

### VARIETY DENOMINATION

‘OLL-8’

### BACKGROUND OF THE INVENTION

The present invention relates to a new and distinct variety of sweet orange tree named ‘OLL-8’. ‘OLL-8’ is a somaclone regenerated from embryogenic callus (tissue culture) of ‘Orie Lee Late’ (‘OLL’) sweet orange (unpatented), with high-quality fruit that usually matures in the standard ‘Valencia’ (unpatented) time period. ‘OLL’ is believed to be an irradiation-induced mutant line originating from Pineapple sweet orange in an abandoned USDA trial attempting to generate a seedless Pineapple orange. ‘OLL’ attracted attention because it never dropped fruit, a serious problem with Pineapple, and held quality late into the summer. Propagations of ‘OLL’ were not uniform and included some trees of poor growth and productivity, as well as robust high-yielding trees. In efforts to generate genetically stable clones from the ‘OLL’ selection, tissue cultures (embryogenic callus) were established for the creation of somaclones. ‘OLL-8’ was the most precocious bearing tree among the population of somaclones regenerated. Trueness-to-type of ‘OLL-8’ was demonstrated by topworking two trees to Swingle citrumelo rootstock in CREC Block 18-North 40. Topworked trees are stable and productive, producing true-to-type fruit (for 4 consecutive years). In addition, young trees propagated on various rootstocks are stable and produce true-to-type fruit at multiple trial locations.

### SUMMARY OF THE INVENTION

Sweet orange selection ‘OLL-8’ produces round oranges with internal and external color significantly higher than that of standard ‘Valencia’ and often exceeds that of ‘Rhode Red Valencia’ (Table 1). Fruit holds on the tree exceptionally well, and maintains quality into the summer. ‘OLL’ seedling trees (including ‘OLL-8’) have shown good drought tolerance in the greenhouse, as overgrown trees in small pots rarely wilt, compared to other sweet orange seedlings, and ‘OLL’ somaclone field trees in general have shown better cold-tolerance in the previous two freezes than ‘Valencia’ trees in the same trial block. This apparent improved stress tolerance may be useful in the fight against Huanglongbing disease. Although minimal formal yield data is available, trees of ‘OLL-8’ appear to yield better than standard ‘Valencia’. The original ‘OLL-8’ tree yielded more than five 90-lb boxes of fruit in 2012 (8-year old tree). Sensory (taste panel) analyses have repeatedly indicated that ‘OLL-8’ has a favorable ‘Valencia’-like flavor profile. Therefore, ‘OLL-8’ has potential to improve the not-from-concentrate (NFC) portfolios of citrus juice processors. ‘OLL-8’ fruit generally has high juice content and produces a good number of pounds of solids. ‘OLL-8’ peels easier than a standard ‘Valencia’, but still requires quadrant scoring for clean peeling when grown under Florida conditions. With its added color, ‘OLL-8’ could also be a valuable addition to the Florida fresh market portfolio.

Fruit of ‘OLL-8’ cannot be distinguished from the fruit of the original ‘OLL’ tree. However, the ‘OLL-8’ tree itself represents a significant improvement, since it has beneficial traits that can be stably propagated, whereas the tree ‘OLL’ cannot.

TABLE 1
Comparison of ‘OLL-8’ with Selected Varieties

| | May, 2008 | June, 2008 | April, 2012 |
|---|---|---|---|
| ‘Valencia’ | 37.31 | 39.90 | 37.47 |
| ‘Rhode Red | 38.21 | 40.60 | 38.60 |
| Valencia’ | | | |
| ‘OLL-8’ | 40.52 | 41.80 | 39.62 |

### BRIEF DESCRIPTION OF THE DRAWINGS

‘OLL-8’ is illustrated by the accompanying photographs, which show the tree's form, foliage and fruit. The colors shown are as true as can be reasonably obtained by conventional photographic procedures.

FIG. 1—Shows a close-up of the mature fruit with the rind present and cross-sectional view of the fruit when cut in the center (February, 2011).

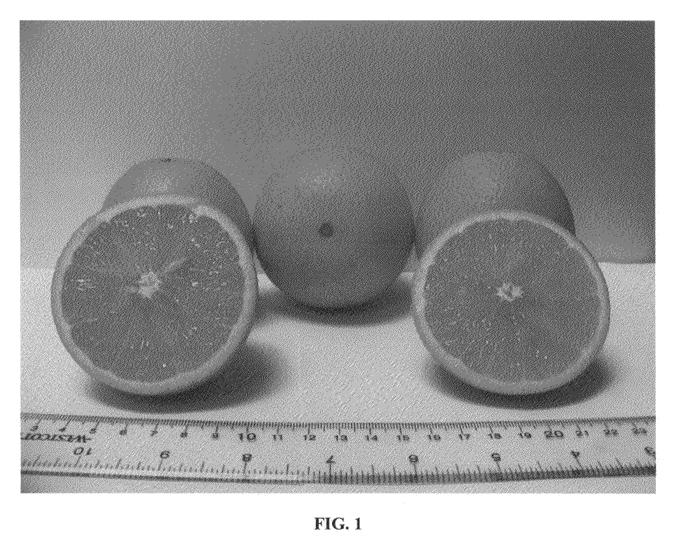

FIG. 2—Shows the original ‘OLL-8’ tree on Swingle citrumelo rootstock, planted East of St. Cloud, Fla. (February, 2011).

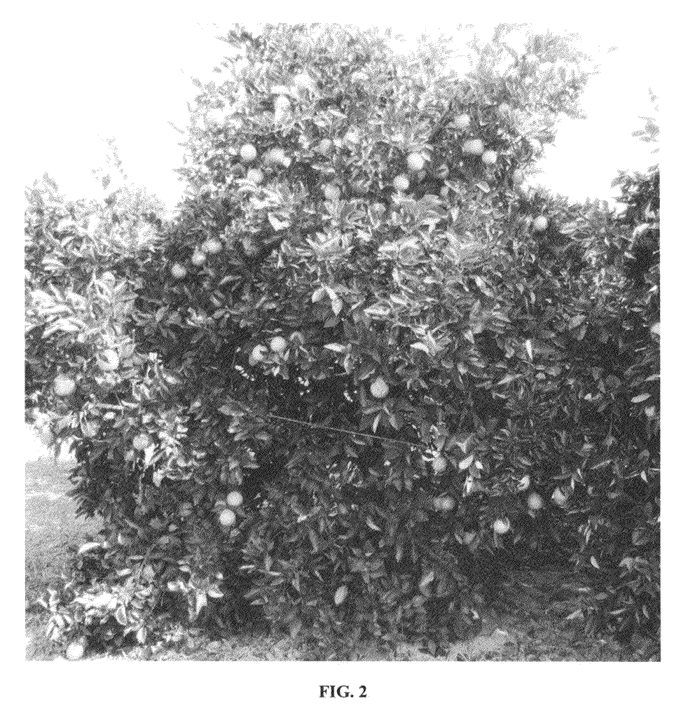

FIG. 3—Shows a top-worked tree of ‘OLL-8’ onto Swingle citrumelo in Lake Alfred, Fla. (February, 2011).

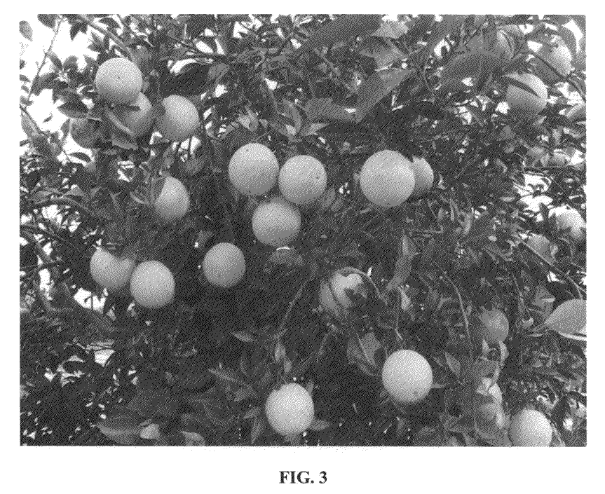

FIG. 4—Shows a close-up view of ‘OLL-8’ sweet orange fruit (February, 2011).

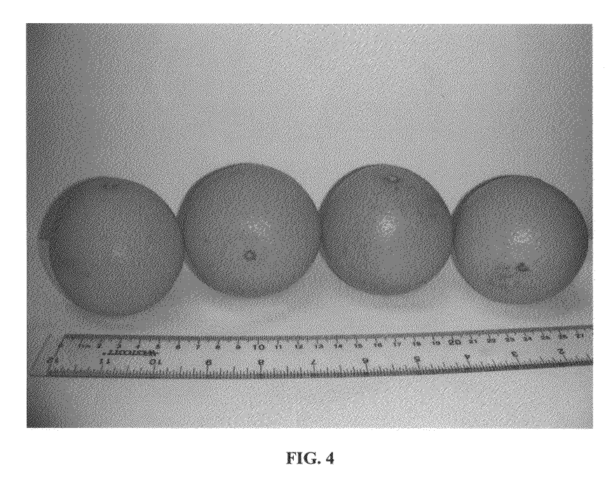

### DETAILED BOTANICAL DESCRIPTION

### Phenotypic Description of Citrus sinensis ‘OLL-8’

Classification:
   Botanical.—Citrus sinensis.
   Common name.—Sweet orange.
Parentage: ‘OLL’ (‘Orie Lee Late’) sweet orange tree, believed to have originated from a 40+year old abandoned USDA irradiated Pineapple sweet orange trial.
Tree:
   Ploidy.—Diploid.
   Size.—Medium.
   Tree height.—3.4 to 3.6 m.
   Tree spread.—3 to 3.2 m.
   Vigor.—Vigorous.
   Density.—Canopies are quite dense.
   Form.—The shape of the tree is obloid with upright and lateral branches growing, but tending toward medium angles after fruit has commenced.
   Growth habit (current season).—Drooping.
Trunk:
   Trunk diameter.—10 to 11 cm at 30 cm height above the ground.
   Trunk texture.—Smooth.
   Trunk bark color.—RHS 197A (greyed-green), irregularly striated with RHS N200 (brown).
Branches:
   Branch length.—2.8 to 3 m from first crotch to the tip of leaf branch.
   Branch texture.—Relatively smooth with small thorns or spines.
   Branch color (shoots from previous flush, hardened, and 4 to 5 mm in diameter).—RHS 138A (green).
Leaves:
   Size (lamina average).—Length: 105 mm Width: 48 mm.
   L/W ratio.—2.1 to 2.2.
   Thickness.—Regular and average for commercial mandarin hybrids.
   Type.—Simple.
   Shape.—Elliptical.
   Apex.—Slightly retuse.
   Base.—Acute to sub-obtuse.
   Margin.—Entire, slightly undulate.
   Surface.—Upper surface: Glabrous Lower surface: Medium veins that are pinnately netted.
   Color.—Upper surface (adaxial): RHS 137A (green) Lower surface (abaxial): RHS 137C (green).
   Petiole.—Shape: Brevipetiolate (shorter than leaf lamina); junction between petiole and lamina is articulate. Width (petiole wing): Narrow Shape (petiole wing): Obovate Length: 13 to 15 mm Width: 2.8 to 3.5 mm Color: RHS 137A (green).
Flowers and flower buds:
   Type.—Hermaphrodite.
   Flower bearing (single or cluster).—Flowers grown from leaf terminals and leaf axillaries. Some branches have flowers grown in clusters, each cluster consisting of 2 to 6 flowers.
   Diameter.—Fully open flower with diameter ranging from 30 to 32 mm.
   Depth.—Typical flower depth, between 19.5 to 20 mm.
   Blooming period.—First bloom: Observed around mid to late March. Full bloom: Observed in early April of 2010.
   Flower bud size.—Length: 2.0 mm for the initial visible flower bud; 18.7 mm for mature flower bud before fully open. Diameter: 2.2 mm for the initial visible flower bud; 5.5 to 6 mm for mature flower bud before fully open.
   Flower bud shape.—Round dome shaped for initial visible flower bud; mature flower bud with elongated olive shape.
   Color.—RHS 144D (yellow-green) for initial visible flower bud.
   Fertility.—Self-fertile.
   Flower petals.—Number: 5 Length: 21.0 to 22.0 mm on average Width: 8.0 mm on average Shape: Flat spatula shaped Apex shape: Smooth acute shaped Base shape: Even obtuse.
   Color.—Upper surface: RHS 155D (white) to RHS 155C (white) Lower surface: RHSNN 155A (white) with RHS 150D (yellow-green) spots distributed toward the petal apex Margin: Smooth.
   Sepal.—Number (per flower): 3 to 5 per flower Shape: Short flat delta with acute angle at apex Length: 2.8 mm Width: 1.8 to 3.6 mm Apex shape: Acute angle at apex Margin: Smooth.
   Color.—Upper surface: RHS 157B (green-white) Lower surface: RHSN157B (green-white).
   Pedicel.—Number: 5 Length: 7 to 7.8 mm Diameter: 0.8 to 1.0 mm Color RHS: RHS 144D (yellow-green).
   Stamen.—Length: 13 to 14.8 mm.
   Anther.—Length: 3.0 to 3.2 mm Width: 1.0 mm.
   Pistil.—Number: 1 Length: 12.2 mm.
   Style length: 8.9 mm.
   Style diameter.—0.8 to 1 mm.
   Style color RHS.—RHS 144D (yellow-green).
   Ovary shape.—Oval shaped.
   Ovary diameter.—2.6 mm.
   Ovary color RHS.—RHSNN144D (yellow-green).
   Blossom period.—In late March to early April in Central Florida.
   Fragrance.—Fragrant.
Fruit:
   Fruit weight.—180 to 205 g on average.
   Size.—Uniform.
   Diameter.—74.4 mm on average.
   Length.—73.6 mm on average.
   Shape.—Round and earth-shaped.
   Shape (cross-section).—Round.
   Apex.—Truncated with shallow dent mark.
   Base.—No neck, with slightly rough peel surface.
Skin:
   Adherence.—Albedo (mesocarp) to fresh (endocarp) is strong and relatively difficult to separate.
   Thickness.—3 to 4 mm, thin to medium compared to other commercial sweet orange varieties.
   Texture.—Firm.
   Color.—Flavedo (epicarp): RHS 23A (yellow-orange) Albedo (mesocarp): RHS 16C (yellow-orange) Stylar end: Closed.
   Oil cell density.—176 to 180 oil cells per square centimeter.
Flesh:
   Number of segments.—9 to 10 segments per fruit on average.
   Segment walls.—Soft, but of sufficient strength to maintain integrity as separated.
   Juice.—Abundant.
   Color.—Uniformly RHS 25B (orange).
   Texture.—Soft.
   Vesicles.—Medium thickness Length: 14.2 to 14.4 mm on average Width: 2.5 to 2.6 mm on average.
   Eating quality (data from late April, 2008).—Sucrose content (average Brix): 13.7 Acidity (average): 0.85 Sugar acid ratio: 15.5.
Seeds:
   Type.—Polyembryonic.
   Number.—Ranges from 1 to 5, usually 4 to 5.
   Shape.—Variable: clavate/club shaped to deltoid shaped.
   Size.—Length: 2.5 to 4.5 mm on average. Width: 7.5 to 9 mm on average.
   Seed coat color.—Outer Surface: RHS 155A (white) and slightly wrinkled Inner surface: RHS 165C (greyed-orange).
   Cotyledon color.—RHS 155A (white).
Resistance to disease: No obvious tree-debilitating disease problems have been observed in the trees or fruit currently grown, but systematic resistance testing has not been explored. Citrus canker lesions have been found on leaves and a few fruit of somaclone ‘OLL-8’, suggesting that its canker tolerance is less than that of ‘Valencia’, and probably more similar to that of ‘Hamlin’.

What is claimed is:

1. A new and distinct cultivar of sweet orange tree as illustrated and described herein.
